(19) United States
(12) Patent Application Publication
(10) Pub. No.: US 2026/0096836 A1
(43) Pub. Date: Apr. 9, 2026

(54) SACROILIAC JOINT FIXATION AND FUSION IMPLANTS

(71) Applicant: GLOBUS MEDICAL, INC., AUDUBON, PA (US)

(72) Inventors: David Peretz, Wynnewood, PA (US); Robert Choh Fleming, Wynnewood, PA (US); Weston Carpenter, Glenmoore, PA (US); Andrew Fluck, Gilbertsville, PA (US); Caelan Allen, Ambler, PA (US)

(21) Appl. No.: 18/905,615

(22) Filed: Oct. 3, 2024

(51) Int. Cl. A61B 17/70; A61B 17/86; B22F 10/28; B22F 10/66; B33Y 10/00; B33Y 40/20; B33Y 80/00

(52) U.S. Cl. CPC A61B 17/7032; A61B 17/1604; A61B 17/8605; A61B 17/863; A61B 17/866; B33Y 10/00; B33Y 40/20; B33Y 80/00; B22F 10/28; B22F 10/66

(57) ABSTRACT

Bone implants, assemblies, and methods thereof. The implants may include a tulip head and a bone fastener including a screw shank with bone threads, a distal tip configured to facilitate insertion into bone, and a proximal end having a screw head receivable in the tulip head. The screw shank may define one or more longitudinal windows and/or helical cuts filled with a lattice structure that acts as a scaffold for bone healing and bone interdigitation. The bone fastener, or a portion thereof, may be 3D printed, for example, using an additive laser powder bed fusion process to provide the integrated lattice structure.

### FIELD OF THE INVENTION

The present disclosure relates to surgical devices, and more particularly, to implants and methods for fixating and/or fusing a sacroiliac joint.

### BACKGROUND OF THE INVENTION

There are two common techniques for fixating long constructs to the pelvis: traditional sacral-alar-iliac (SAI) and iliac screws or bolts. Both types of fixation may be used as an anchor for a pedicle screw construct and are typically used in longer deformity cases to provide additional stability and to help offload the S1 screws. Current uses include long constructs, as well as high-grade spondylolisthesis, unstable sacral fractures, and others. Each of these require the additional support that SAI and iliac screws provide to ensure a secure lumbosacral foundation that can withstand the forces acting on constructs at the L5/S1 junction. While SAI and iliac screws are used to support these thoraco-lumbar or longer constructs, SAI screws are shown to also aid in reducing sacroiliac joint (SIJ) pain by limiting range of motion (ROM). Surgeons have seen that patients exhibiting SIJ pain and requiring sacro-pelvic fixation may benefit from a screw that can achieve SIJ fusion. As such, there exists a need for implants that provide acute fixation and long-term fusion of the SIJ, while having the capabilities to integrate with long constructs including rods.

### SUMMARY OF THE INVENTION

To meet this and other needs, implants, assemblies, and methods are provided. In particular, the sacroiliac joint may be fixated and/or fused via a threaded implant that may mount rigidly to a fixating rod or rigid connector. For example, the threaded implant may serve as a foundational anchor for a pedicle screw and rod construct. The implant may include a bone fastener with a threaded screw shank having an integrated lattice structure configured to promote bony ingrowth and improve the bone interface strength. The lattice structure may extend through one or more transverse windows through the screw shank and/or within helical cuts along the body of the screw shank. The bone fastener may be printed with a three-dimensional (3D) additive process, in whole or in part, for example, using an additive laser powder bed fusion process for creating the integrated lattice structure to increase the likelihood of fusion. The bone threads may also be optimized for 3D printing and to ensure bone fixation in multiple loading scenarios. These implants may be used in bilateral, open, and percutaneous approaches to the spine and/or ilium and may be compatible with robotic and/or navigation systems.

According to one embodiment, a sacroiliac implant includes a tulip head having two arms defining a rod slot therebetween, and a bone fastener extending along a central longitudinal axis including a screw shank with bone threads following a helical path, a distal tip configured to facilitate insertion into bone, and a proximal end having a screw head receivable in the tulip head. The screw shank defines first and second longitudinal windows extending therethrough. The first and second longitudinal windows are staggered and shifted longitudinally and rotationally relative to one another. The screw shank further defines a helical cut that is non-coincident with the helical path of the bone threads such that the helical cut interrupts at least some of the bone threads. The first and second longitudinal windows and the helical cut are filled with a lattice structure configured for promoting bone ingrowth.

The sacroiliac implant may include one or more of the following features. The first and second longitudinal windows may be rotated 90 degrees around the central longitudinal axis of the screw shank relative to one another. The first and second longitudinal windows may be obround slots. The first and second longitudinal windows may intersect such that peripheries of the windows overlap with one another.

The helical cut may include a first distal helical segment that overlaps a portion of the first longitudinal window and second proximal helical segment that overlaps a portion of the second longitudinal window. The pitch of the helical cut may be greater than the pitch of the bone threads. The depth of the helical cut may be shallower than the root of the screw threads such that a portion of the screw threads remain. Alternatively, the depth of the helical cut may be equivalent or deeper than the root of the screw threads such that the screw threads are completely eliminated or erased along the helical cut. The screw threads may include an asymmetrical profile with a sloped leading edge and a flat trailing edge, for example, resembling tapered buttress threads. The lattice structure may include interconnected struts defining open pores of different sizes, for example, having a geometry similar to cancellous bone to promote fusion and bone growth within the screw shank.

According to one embodiment, a sacroiliac implant includes a tulip head and a bone fastener including a screw shank with bone threads. The bone threads may have a deep root and an internal taper. The major outer diameter may be constant while the minor diameter is tapered along its length with an increasing thread root size resulting in the same screw outer diameter throughout its entire length. The screw shank may define first and second longitudinal windows extending therethrough that are staggered and shifted longitudinally and rotationally relative to one another. The windows are filled with a lattice structure configured for promoting bone ingrowth. Alternatively, the screw shank may have a less extreme internal taper resulting in a less extreme thread depth, which may allow the bone fastener to act as a wedge to promote increased initial fixation before fusion can occur.

According to one embodiment, a sacroiliac implant includes a tulip head and a bone fastener including a screw shank with bone threads. In this embodiment, in addition to the lattice-filled longitudinal windows, the implant includes a distal lattice tip. The distal lattice tip may include an internal lattice area provided on the inside and along the shank, but not on the threads of the screw shank.

The lattice structure at the distal tip may be revealed through machining after the 3D printing process, while leaving the crests of the threads in solid form. Alternatively, the lattice tip may encompass the entire thread including the thread crests as well. This distal lattice tip structure may be formed during the 3D printing process, requiring no further processing.

According to one embodiment, a sacroiliac implant includes a bone fastener assembled from three distinct parts: a solid tip, a lattice core, and a solid base. The solid tip includes a distal tip portion with bone threads. The proximal end of the distal tip portion includes an extension positionable through the lattice core and into the solid base. The lattice core may include a ring of lattice matrix. The lattice core may be 3D printed, for example, using the additive laser powder bed fusion process to provide the lattice structure through its body. The solid base may include a proximal portion of the shaft with bone threads. An assembly pin may be configured to secure the extension of the distal tip portion, including the lattice core around the extension, within the solid base. Alternatively, the components may be welded or otherwise secured together.

According to one embodiment, a sacroiliac implant includes a bone fastener including a screw shank with bone threads. The bone threads include dual lead threads with a first solid-filled thread and a second lattice-filled thread in an alternating pattern. In this embodiment, the lattice runs along the entire length of the screw shank along one of the thread grooves to allow for sufficient bone in-growth and fusion.

According to one embodiment, a manufacturing process may include: (a) applying a layer of fine metal powder to a build plate of a three-dimensional printing machine and selectively sintering metal in prescribed locations to create a layer of a screw part blank; and (b) consecutively adding and sintering layers of fine metal power to prior layers to build up the screw part blank. The screw part blank includes a screw shank with solid and lattice portions. The solid portion includes the bone threads, and the lattice portions fill first and second longitudinal windows and/or a helical cut about a periphery of the screw shank. The first and second longitudinal windows may be staggered and shifted longitudinally and rotationally relative to one another. If present, the helical cut may not align with a helical path of the bone threads, thereby interrupting some of the bone threads where the paths intersect. The process may also include creating a penholder at a distal tip of the screw part blank when adding and sintering the layers of metal powder. The penholder may include a cone of solid material deposited around the distal tip of the screw part blank. The process may include creating space at the distal tip relative to the penholder for easy removal of the screw part blank from the penholder. The bone threads may include a tapered buttress style geometry where inter-thread support material is not needed and the threads are able to self-support during the entire manufacturing process. The three-dimensional printing machine may include a laser powder bed fusion machine or other suitable 3D printing process. The process may also include machining the screw part blank to form the screw head and drive recess, thereby creating the final bone screw. If desired, the bone threads may also be machined to precise specifications.

According to one embodiment, a method for stabilizing a sacroiliac joint may include: (a) providing an implant having a screw shank with bone threads, the screw shank defining first and second longitudinal windows extending therethrough that are staggered and shifted longitudinally and rotationally relative to one another, the screw shank further defining a helical cut that has a pitch greater than a pitch of the bone threads such that the helical cut interrupts at least some of the bone threads, wherein the first and second longitudinal windows and the helical cut are filled with a lattice structure that acts as a scaffold for bone healing and bone interdigitation; (b) accessing a sacrum and/or ilium of a patient; and (c) inserting the implant across the sacroiliac joint such that once the implant is fully seated, the first and second longitudinal windows engage with the sacrum and ilium, respectively, thereby traversing the sacroiliac joint at final placement to increase the likelihood of fusion. The first longitudinal window may be a distal window configured to be positioned in the ilium, and the second longitudinal window may be a proximal window configured to be positioned in the sacrum, and an intersection of the windows may provide for in-growth capabilities through the lattice structure, further promoting fusion. The method may include installing a pair of implants, which are used as bilateral S2-alar-iliac screws to fix the sacrum to the ilium in a lumbosacral fixation. When secured to spinal rods, the bilateral implants may function as anchors for the pedicle screw and rod constructs. The method may include accessing the sacroiliac joint or performing other surgical tasks with a robotic and navigational system.

According to one embodiment, a method for stabilizing a sacroiliac joint may include: (a) providing one or more implants of the types described herein; (b) accessing a sacrum and/or ilium of a patient through a lateral approach or a posterior approach (e.g., lateral to medial or medial to lateral); and (c) inserting the implant across the sacroiliac joint, thereby providing fixation and promoting fusion of the two bones. Multiple implants may be inserted across the joint to better stabilize and prevent movement of the sacroiliac joint. The anatomy of the patient may be accessed using a standard or minimally invasive surgical (MIS) technique. The surgery may be performed with the assistance of robotic and/or navigational systems.

Also provided are kits including implants of varying types and sizes, bone fasteners, spinal rods, k-wires, insertion tools, instruments, bone cement, biomaterials, and other components for performing the procedure(s).

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIGS. 1A-1D show perspective, side, and top views, respectively, of a sacroiliac implant having a 3D printed threaded screw shank with an integrated lattice structure and a polyaxial tulip head according to one embodiment;

FIGS. 2A-2C show the sacroiliac implant highlighting regions of internal lattice and a helical outer channel according to one embodiment;

FIGS. 3A-3B further show implant having transverse windows filled with internal lattice portions according to one embodiment;

FIG. 4 shows a close-up perspective view of the proximal end of the threaded screw with transverse windows filled with lattice structure according to one embodiment;

FIGS. 5A-5B demonstrate penholder print examples for a laser power bed fusion process with minimal support material used on the distal tip to build the part according to one embodiment;

FIGS. 6A-6B show cross-sections of the implant with bone threads designed for additive manufacturing and screw-bone interaction according to one embodiment;

FIG. 7 is a 3D additive laser powder bed fusion manufacturing process workflow according to one embodiment;

FIGS. 8A-8C show cross-sections and a perspective view, respectively, of a deep root screw with an internal taper and lattice windows according to one embodiment;

FIG. 9 shows a perspective view of a tapered screw with lattice-filled windows and a large internal taper according to one embodiment;

FIGS. 10A-10C show cross-sections and a perspective view, respectively, of a threaded screw with lattice-filled windows and an internal lattice at the distal tip according to one embodiment;

FIGS. 11A-11B show a threaded screw with lattice-filled windows and a full lattice distal tip and a blank of the implant with a penholder support created using laser powder bed fusion according to one embodiment;

FIGS. 12A-12D show cross-sectional, perspective, and axial views, respectively, of a threaded screw with lattice-filled windows and a continuous helical cut filled with lattice according to one embodiment;

FIGS. 13A-13B show cross-sectional and perspective views, respectively, of examples of threaded screws with a lattice distal tip;

FIGS. 14A-14C show cross-sectional and perspective views, respectively, of a threaded screw with lattice-style windows, and a blank of the implant created via the laser powder bed fusion additive process with a penholder tip according to one embodiment;

FIGS. 15A-15C show a three-part implant assembly with an inner lattice core ring according to one embodiment; and

FIGS. 16A-16C show cross-sectional and perspective views, respectively, of a double thread start with lattice uniformly distributed along one lead of the screw, and a close-up view of the lattice section between solid threads according to one embodiment.

### DETAILED DESCRIPTION OF THE INVENTION

Implants, assemblies, and systems are configured to fixate and/or fuse the sacroiliac joint. The implants may include threaded shanks, which may be 3D printed with internal lattice structures that are configured to promote bone fixation and/or prophylactically fuse the sacroiliac joint. The threaded shanks may include integrated solid and lattice areas, which maintain strength, optimize stress distribution, and promote better integration of the screw with surrounding bone tissue. The 3D printing process may allow for an open channel design with intersecting windows and an integrated lattice structure to provide more bone in-growth opportunities. The lattice structures within the screw can act as a scaffold for bone growth and increase the surface area, which can enhance the biological interface between the screw and bone. The bone implants may be used independently or may include the capability to integrate with long rod constructs, for example, with a tulip or other suitable attachment interface, to anchor the rod construct in the sacroiliac joint.

These implants may be used in bilateral, open, and percutaneous approaches to the spine and/or ilium and may be compatible with robotic, imaging, and/or navigation systems. Details of robotic and/or navigational systems can be found, for example, in U.S. Pat. Nos. 10,675,094, 9,782,229, and U.S. Patent Publication No. 2017/0239007, which are incorporated herein by reference in their entireties for all purposes.

Although described herein with reference to the sacroiliac joint, it will be appreciated that the devices described herein may be applied to other areas of the spine, other orthopedic locations in the body, and other medical procedures, such as trauma applications. Any of the implants described herein may be offered in a multitude of styles, sizes, and lengths, helping to ensure optimal patient fit. The 3D printing process allows for the creation of screws specifically designed to meet the unique anatomical and mechanical needs of individual applications or patients. Each screw can be customized in terms of size, thread pattern, and the distribution of solid and lattice areas, for example.

The implants or components thereof may be comprised of titanium, stainless steel, cobalt chrome, cobalt-chrome-molybdenum, tungsten carbide, carbon composite, plastic or polymer—such as polyetheretherketone (PEEK), polyethylene, ultra-high molecular weight polyethylene (UHMWPE), resorbable polylactic acid (PLA), polyglycolic acid (PGA), allograft, autograft, or combinations of such materials or any other appropriate material that has sufficient strength to be secured to and hold bone, while also having sufficient biocompatibility to be implanted into a body. Although the above list of materials includes many typical materials out of which implants may be made, it should be understood that implants comprised of any appropriate material are contemplated.

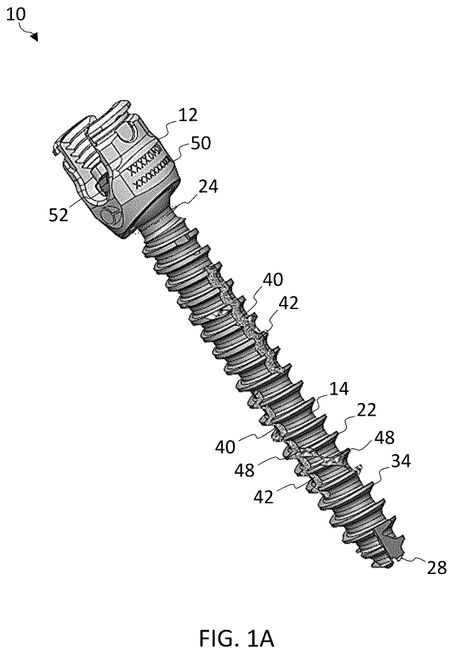

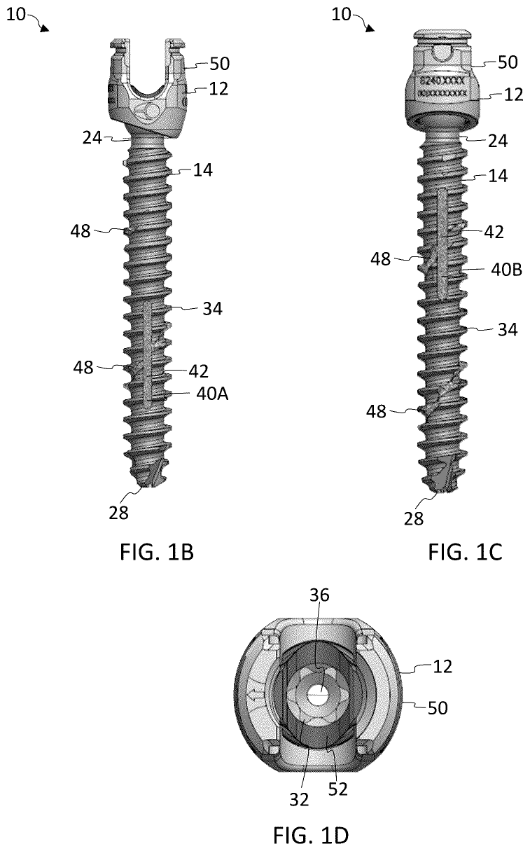

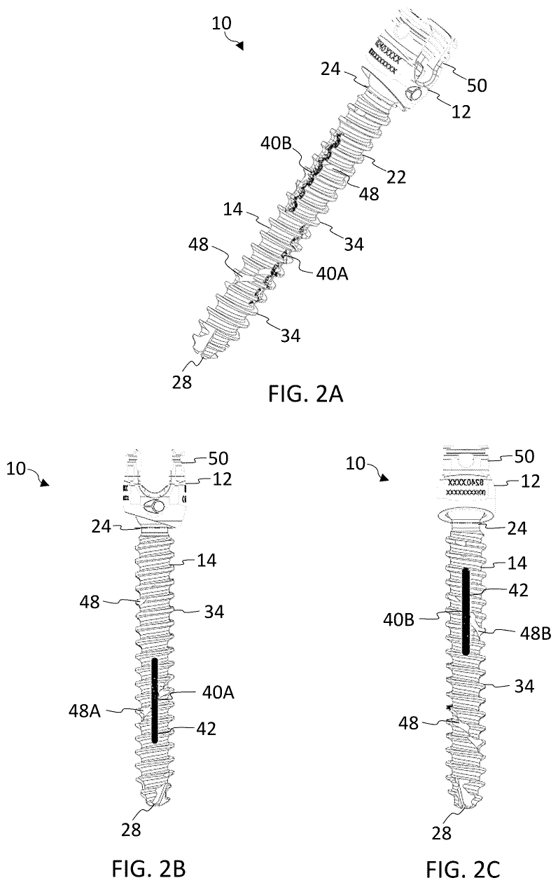

Turning now to the figures, where like reference numbers may refer to like elements, FIGS. 1A-1D shows an orthopedic fixation device or sacroiliac implant 10 according to one embodiment. The sacroiliac implant or bone fastener assembly 10 may include a screw head or tulip head assembly 12 attachable to a bone fastener 14, which are configured to anchor a spinal rod for fixation. The bone fastener 14 may be included in an assembly with pre-assembled tulip heads 12 of varying styles, or as a modular component where a modular head assembly is attached to the bone fastener 14 intraoperatively. For a polyaxial implant 10, the tulip head assembly 12 may permit polyaxial movement relative to the bone fastener 14. The tulip head 12 is configured to receive a spinal rod and a locking cap, which secure the spinal rod therein. For a polyaxial bone fastener, tightening the locking cap compresses the rod into the tulip head 12, thereby restricting motion of the bone fastener 14 and forming a rigid construct. In addition to polyaxial tulip head styles, it will be appreciated that any suitable tulip assembly may be selected including unilateral, monoaxial, fixed, reduction, etc., which offer different degrees of flexibility, stability, and ease of use based on the requirements of the spinal procedure. Examples of bone fasteners, other implants, and rod constructs are described in more detail, for example, in U.S. Pat. No. 10,368,917, which is incorporated by reference herein in its entirety for all purposes.

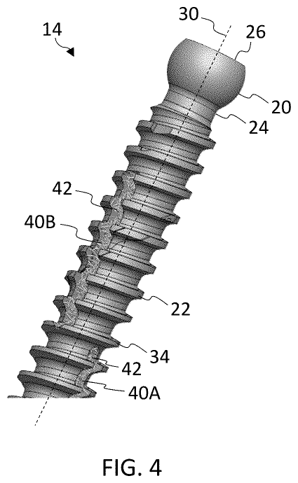

The bone fastener 14 may include a bone screw, anchor, clamp, or the like configured to engage bone. As best seen in the close-up view in FIG. 4, the bone fastener 14 may include a bone screw having a screw head 20 connected to a threaded shaft 22 by a neck 24. The bone fastener 14 extends from a proximal end 26 to a distal end 28 along a central screw axis 30. While the screw head 20 may have any general shape, in the case of a polyaxial fastener 10, at least a portion of the screw head 20 may have a curved surface in order to allow for rotational movement and/or angular adjustment of the bone fastener 14 with respect to the tulip head 12. For example, at least a portion of the screw head 20 may be shaped to form a portion of a ball or at least a portion of a sphere. The screw head 20 may be smooth, threaded, provided with a roughened or textured surface, or may be otherwise configured to interface with the tulip head assembly 12. The screw head 20 may have a tool engagement surface or drive recess 32 that can be engaged, for example, by a screw-driving instrument or other device. The drive recess 32 is housed in the top of the screw head 20. In one embodiment, the drive recess 32 has a hexalobe shape for driving the screw 14 into bone. It will be appreciated that any suitably shaped tool engagement surface may be provided.

The threaded shaft 22 includes one or more bone threads 34 configured to engage bone. The bone threads 34 include external helical ridges that follow a helical path around the periphery of the shank 22, which are configured for anchoring the bone fastener 14 into bone. Varying bone thread forms may be used, such as corticocancellous, dual outer diameter (DOD), or cortical (e.g., midline cortical screw or MCS). The bone threads 34 may include a single lead with one continuous thread that spirals around the screw's body. The bone threads 34 may include dual lead threads with two separate leads spiraling around the screw shaft starting at different points, which may help the screw 14 to advance faster into the bone. It will be further appreciated that the bone threads 34 may include other variations, such as triple lead threads, variable pitch threads, fluted threads, etc. The threaded shaft 22 may have a number of different features, such as thread pitch, shaft diameter to thread diameter, overall shaft shape, and the like, depending, for example, on the particular application. The designs may be tailored to meet specific biomechanical needs, optimize bone healing, enhance surgical outcomes, and reduce insertion times, for example. In one embodiment, the back end of the screw shank 14, towards the tulip end, may be designed with a slightly thicker minor diameter for increased bone purchase during insertion, especially when inserted into a cannulated hole of a constant diameter. The proximal end of the screw 14, near the tulip 12, may also include reverse cutting teeth to facilitate bone cutting during revision surgery if necessary. The neck 24 of screw 14 may also be thicker than other screws for an increase in screw robustness.

Cannulations and fenestrations may also be employed for placement over a guide wire or k-wire and/or for delivery of bone cement. In one embodiment, as best seen in a top-down view from the tulip head 12 in FIG. 1D, the bone fastener 14 may be optionally cannulated 36 along the screw axis 30 all the way through the implant 10. The cannulation 36 may run longitudinally along the central screw axis 30 through the entire length of the implant 10 to accommodate a guide wire for increased surgical precision.

The threaded shaft 22 terminates at the distal end 28 as a distal tip. As shown in FIG. 1A, the distal tip 28 may be generally blunt to prevent damage to soft tissue. Alternatively, the distal tip 28 may be pointed or may include cutting edges around the cannulation 36 to aid in starting the screw 14. It will be appreciated that varying tip geometries may be tailored for specific applications. The screws 14 may be made in a variety of diameters and lengths to match the anatomy of the patient. The screw 14 may also optionally undergo a hydroxyapatite (HA) coating, surface treatment, or other additive processes if desired.

The screw shaft 22 defines one or more windows or fenestrations 40 extending through its body. The windows 40 may include transverse longitudinal slots or graft windows, which are oriented along the axis 30 of the screw 14. The windows 40 are longitudinally oriented and run parallel to the axis 30 of the screw. Each window 40 may have a width, for example, no greater than the diameter of the cannulation 36. Each window 40 may have a length that is substantially longer than its width. The length may be, for example, about one quarter or more or one third or more of the length of the screw 14. The windows 40 may be shaped like elongated ovals, obround slots, or rounded rectangular forms. The elongated slots may help to maximize the window area without compromising the structural integrity of the screw shaft 22. The rounded edges at the proximal and distal ends of the window 40 may help to reduce stress concentrations. The windows 40 may be defined into the cylindrical body of the screw shank 14 and extend through its diameter, allowing for fluid communication from one side to the other.

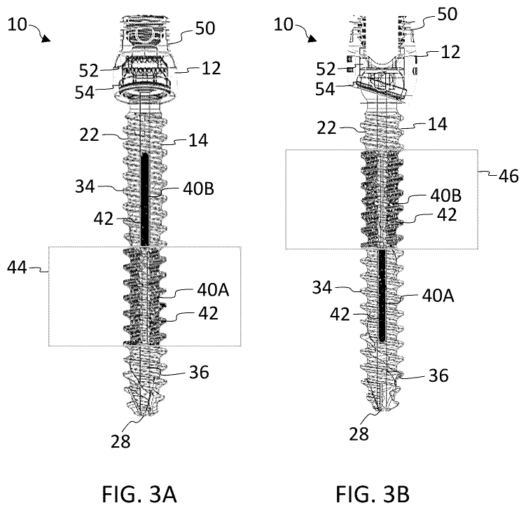

Each window 40 may have a longitudinal placement distributed along the length of the screw's body. The windows 40 may be staggered and shifted longitudinally and/or rotationally. The windows 40 may be spaced at regular intervals and offset relative to one another around the circumference of the screw 14. For example, successive windows 40 may be rotated approximately 90 degrees around the axis 30 of the screw relative to a previous window 40. The windows 40 may be shifted longitudinally along the shaft 22 such that a first window 40A is provided distally (e.g., in the lower half of the shaft 22) and a second window 40B is provided proximally (e.g., in the upper half of the shaft 22). As best seen in FIGS. 3A-3B, the first distal window 40A may be oriented along a first plane 44 and the second proximal window 40B may be oriented along a second plane 46 perpendicular to the first plane 44. As shown in FIG. 3A, the implant 10 may be oriented such that the rod axis of the tulip 12 is arranged along the same plane 44 as the distal window 40A and the second plane 46 extends into and out of the page. In FIG. 3B, the implant 10 is rotated 90 degrees and oriented such that the rod axis of the tulip 12 is perpendicular to second plane 46 and the first plane 44 extends into and out of the page.

The offset windows 40 may intersect or partially overlap. For example, the offset windows 40 may partially overlap such that the edges or peripheries of the windows 40 meet or overlap with one another. Although there may be partial overlap, the majority of the windows 40 do not overlap. The intersection of the windows 40 may provide for in-growth capabilities, further promoting fusion. The offset windows 40 may be strategically positioned such that the windows 40 engage with the sacrum and ilium, respectively, thereby traversing the sacroiliac joint at final placement to increase the likelihood of fusion. It will be appreciated that any suitable shape, size, location, and configuration of windows 40 may be provided to promote fusion.

Each window 40 may be filled with a lattice or matrix structure 42 to promote bone ingrowth and osseointegration, enhancing the stability and longevity of the implant 10. In one embodiment, the screw shank 14 may include two intersecting windows 40 with integrated lattice structure 42 that extends throughout the full diameter of the screw 14. The open channels 40 may be strategically placed in order to ensure that architecture traverses the sacroiliac joint at final placement, thereby increasing the likelihood of fusion.

The lattice structure 42 may provide a scaffold with increased surface area for bone healing and bone interdigitation. The lattice structure 42 may have a geometry similar to cancellous bone, which promotes fusion and bone growth within the screw 14. The lattice structure 42 may include a uniform or non-uniform lattice framework. The lattice structure 42 may include a porous scaffold structure, for example, including pores and/or micropores. As the bone heals, the bone grows into the microporous structure further enhancing fixation. In some embodiments, the lattice structure 42 may have grid, honeycomb, hexagonal struts, or other patterns to promote bony in-growth. For example, the lattice 42 may include interconnected struts or beams forming a crisscross or interconnected pattern. The lattice structure 42 may include a randomized or repeating pattern of open or interconnected pores. The lattice structure 42 may also vary in type, size, or porosity, for example, along the length of the implant 10. The pores may be spherical, partially spherical, or of another suitable pore shape or configuration. The lattice structure 42 may have a suitable porosity (open volume), for example, greater than 50% open, greater than 60% open, greater than 70% open. In one embodiment, the lattice structure 42 may have a porosity in the range of about 50-80% to maximize the potential for bony in-growth. The lattice structure 42 may have pore sizes, for example, ranging from approximately 100 μm-2 mm, approximately 100 μm-1 mm, approximately 200-900 μm, or approximately 300-800 μm in diameter. Additional details on suitable lattice or porous structures are described, for example, in U.S. Pat. Nos. 11,534,308 and 10,524,926, which are incorporated by reference herein in their entireties for all purposes.

In one embodiment, the screw shank 14 further defines a helical outer channel, spiral channel, or helical cut 48 along the body of the screw 14. The helical cut 48 may include a continuous or segmented spiral groove or channel cut into the body of the screw 14. In one example, the helical cut 48 may be segmented to include a first distal helical segment 48A and a second proximal helical segment 48B. The helically cut exterior surface 48 may be configured to allow for bone gathering and additional surface area to further encourage fusion.

The helical cut(s) 48 may be non-coincident with the helical path of the screw threads 34. This placement means that the helical cut(s) 48 do not align directly with the natural path of the screw threads 34. In this manner, the helical cut(s) 48 may interrupt the screw threads 34, and the helical cut(s) 48 introduce breaks in the thread pattern. The helical cut(s) 48 may have a different pitch, lead, rotation, translation, scale, or handedness, relative to the screw threads 34. In other words, the helical cut(s) 48 may follow a different path or have a different pitch and/or depth, creating a distinct pattern that disrupts the continuity of at least some of the screw threads 34. In one embodiment, the pitch of the helical cut(s) 48 is greater or larger than the pitch of the screw threads 34. In this manner, the helical cut(s) 48 advance a longer distance along the axis 30 of the screw 14 for each complete turn compared to the screw threads 34. For example, the helical cut(s) 48 may have a greater pitch that overlaps the path, for example, of three or more, four or more, five or more regular threads 34 on the screw's body. With a greater pitch, the helical cut(s) 48 may encourage more material removal and more in-growth opportunities optimizing the biological compatibility.

In addition, the depth of the helical cut(s) 48 may be shallow or less than the root of the screw threads 34. A shallow helical cut 48 may allow for at least a portion of the screw thread 34 to remain intact. For example, the crest of the screw threads 34 may be removed while the remainder of the thread 34 remains. By keeping the helical cut(s) 48 shallow, the structural integrity of the shank 22 and/or threads 34 may be maintained and maximum engagement with the bone may be achieved.

The helical cuts(s) 48 may be configured to overlap a portion of the longitudinal windows 40. For example, the first distal helical segment 48A may overlap the first distal window 40A and the second proximal helical segment 48B may overlap the second proximal window 40B. The helical segments 48, 48B may be configured to overlap the center of each respective window 40A, 40B.

The helical cut(s) 48 may also be filled with lattice structure 42 to promote bone ingrowth and osseointegration. The lattice structure 42 may be the same as the lattice structure within each window 40. Alternatively, a different type or configuration of lattice may be used. The helically cut exterior surface 48 allows for bone gathering to encourage fusion, and the screw's lattice structure 42 also provides in-growth capabilities, further promoting fusion.

As best seen in FIGS. 3A-3B, each screw shank 14 may be combined with a tulip head assembly 12. The tulip head assembly 12 may include a tulip head 50 which houses a saddle 52 and a clip 54. The tulip head 50 includes opposed arms defining a U-shaped channel or rod slot sized and configured to accept the spinal rod. Each of the arms has interior threaded portions for engaging the locking cap such that the spinal rod may be secured in the tulip head 50 with the locking cap. The saddle 52 applies compressive force to the bone screw 14 and restricts its angulation when the rod is tightened to the implant with the locking cap. The clip 54 rests in a groove in the base of the tulip 50 and is configured to fit around a portion of the screw head 20. The clip 54 retains the bone screw 14 within the assembly and resists compressive force exerted down on the bone screw 14.

In one embodiment, the tulip head assembly 12 may be configured as part of a sacral-alar-iliac (S2AI) implant, which enters at the second sacral bone (S2), passes through the alar region of the sacrum, and extends into the ilium (part of the hip bone) to provide pelvic fixation. The S2AI tulip assembly functions similar to a pedicle screw assembly but is configured such that the angulation is preferred in one direction. The tulip head may resist motion in pre-defined directions to allow for correction of deformity in different clinical situations or to allow for better alignment to the spinal rod. To accomplish this, the bottom surface of the tulip 50 and clip 54 may be provided at an angle in the medial/lateral direction with respect to the central axis of the tulip 50. The purpose of the preferred angle is to accommodate the S2AI trajectory in the pelvis, which commonly is at a more extreme and predictable angle when compared to standard pedicle screw trajectories. The tulip 50 may be made from cobalt chrome (CoCr) or titanium alloy, such as titanium aluminum vanadium (TAV), for example, for robust performance. Although a S2AI tulip 12 is shown, it will be appreciated that any suitable tulip assembly may be used with the screw 14. Suitable heads may include polyaxial, modular, reduction, uniplanar, monaxial, open and closed heads options, for example. The implants may be provided pre-assembled reducing the number of steps needed, which simplifies the overall procedure and may reduce operating time. Alternatively, the head assembly may be attached intraoperatively by top loading the tulip head onto the bone screw to provide for modularity of the system.

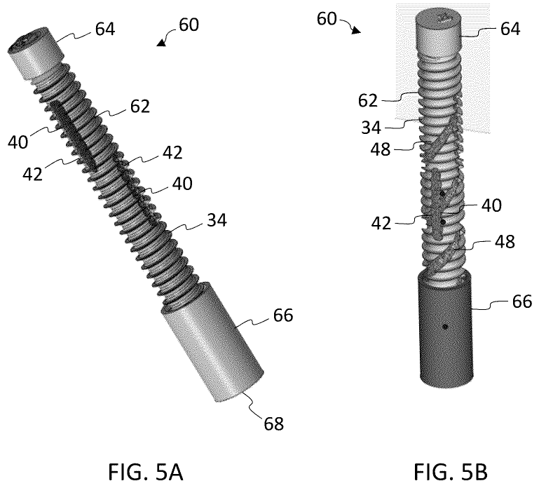

Turning now to FIGS. 5A-5B, the screw shank 14 may be manufactured through 3D printing, allowing for the open channel design 40 with complex lattice structure 42 and the helically cut exterior surface 48 to allow for bone gathering and in-growth to encourage fusion. The specialized bone screw threads 34 are also optimized for 3D printing and ensure bone fixation in multiple loading scenarios.

The screw shank 14 may be created by additive manufacturing, such as three-dimensional (3D) printing. The additive manufacturing may include laser powder bed fusion (LPBF), direct metal laser sintering (DMLS), vat photopolymerization, material jetting, lamination, extrusion, directed energy deposition, or any other suitable additive manufacturing process. In one embodiment, the screw shanks 14 and threads 34 are designed specially to be manufactured using the additive laser powder bed fusion (LPBF) process. Laser powder bed fusion is a technique in which a layer of fine metal powder is deposited, selectively melted, and solidified using a laser to create the solid and/or lattice areas of metal, with consecutive 2D layers being joined to preceding layers to build up the 3D components.

In laser powder bed fusion, parts may be initially welded to a build plate and may include sacrificial support structures. These additional support structures can be large in nature and consume large amounts of raw materials. Finished LPBF builds may need to be cleared of loose powder, heat treated to relieve stresses and improve fatigue life, and then cut from the build plate, for example, using a wire electrical discharge machining (EDM) or a band saw. The part may undergo finish machining and/or surface treatments to complete the final part, and any sacrificial supports need to be cut or broken away as well.

In one embodiment, the screw shank 14 may be printed with the distal tip 28 affixed to the build plate using a penholder approach and a 2-layer thickness (e.g., about 180 um) spacing off of the distal tip 28. This allows the manufacturing process to develop a completed part with no other support material in a way where it can be removed from the support structure and build plate without cutting. This approach may be more efficient in decreasing the build plate cut-off time and support material removal time.

FIGS. 5A-5B show penholder print examples with minimal support material for creating a screw blank 60. The screw model or blank 60 may include a printed shaft 62 with a head portion 64 at the proximal end and a distal support or penholder 66 provided at the distal end. The penholder 66 may include a small cone of solid material that is deposited around the implant body to reduce the amount of required support material and to allow for easier removal from the build plate. The penholder 66 may include a support structure with a distal most face 68 contacting or attached to the build plate or platform of the laser powder bed fusion machine. The proximal end of the penholder 66 may define a conical opening sized and dimensioned to match the shape and size of the distal tip 28 of the implant 10 once fully built. All or portions of the distal tip 28 of the screw 14 may be spaced apart from the conical opening of the penholder 66 during the build for easy removal once completed. During the process, the printed shaft 62 may be produced with the thread design 34, windows 40, helical cut 48, and/or integrated lattice structure 42 of the final shaft 22 of the screw 12.

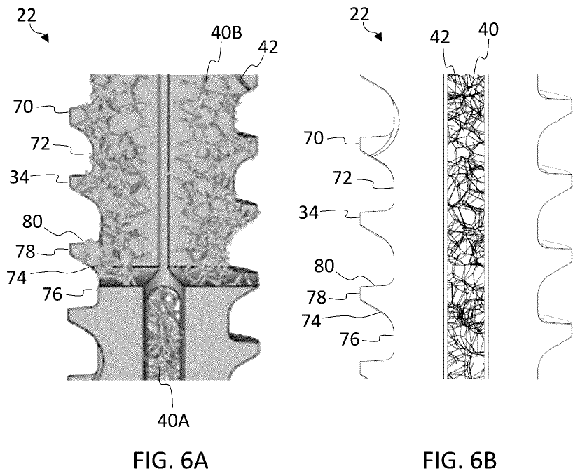

With further emphasis on FIGS. 6A-6B, the screw shank threads 34 are designed for additive manufacturing and screw-bone interaction. In FIGS. 6A-6B, the close-up screw sections are oriented with the tulip head 12 upward and the distal tip 28 downward. The shaft 22 includes a major outer diameter 70 or largest diameter of the thread 34, and a minor inner diameter 72 or the smallest diameter of the thread 34. The bone threads 34 may include an asymmetrical profile, for example, that include a slanted, sloped, or curved leading edge 74 extending from the root 76 to the crest 78 of the thread 34. The trailing edge 80 may include a steep, nearly flat, or planar side face that resembles a sawtooth or a staircase. The screw shank threads 34 may include a tapered buttress style geometry where inter-thread support material is not required and the threads 34 are able to self-support during the entire manufacturing process. Threads can be difficult to form because the melt pool generated in the powder bed is liquid and can flow. The buttress threads 34 saves support material cost and laser scan time. The 3D printed buttress threads 34 also reduce the need for post-op machining of the threads 34 at a later time.

With regard to the screw head 20, the blank head portion 64 may be printed with excess material for later machining. For example, additional material may be added to the spherical head 20 of the screw 14 intended for post-printing machining to obtain proper geometrical tolerancing. For example, the blank head portion 64 may be printed with a cylindrical shape, which may be later machined into the desired spherical cross-section with the drive recess 32 in the proximal face. Alternatively, the head portion 64 may be printed in the final design shape during the 3D printing process.

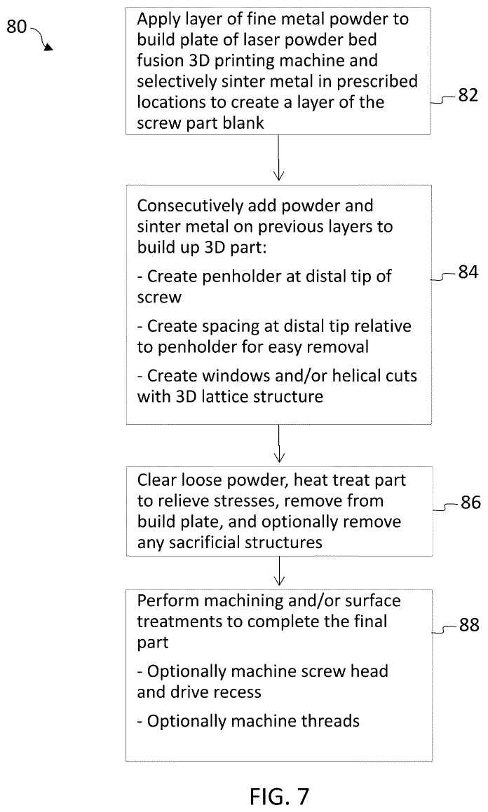

FIG. 7 depicts one example of a 3D additive laser powder bed fusion manufacturing workflow. The screw blank 60 may be planned according to one of the chosen designs, for example, including one or more windows 40 and/or helical cuts 48 with internal lattice structures 42. In a first step 82, a layer of fine metal powder is applied to the build plate of the laser powder bed fusion 3D printing machine. The machine selectively sinters the metal in prescribed locations to create a layer of the screw part blank. In a second step 84, metal powder is applied and sintered, consecutively, on previous layers to build up the 3D part. The sintering steps may create both solid and lattice portions of the screw blank 60, layer by layer, until the screw blank 60 is fully formed. Thus, the sintering steps include creating the windows 40 and/or helical cuts 48 with the integrate 3D lattice structure 42. The process may include, layer by layer, creating the penholder 66 at the distal tip 28 of the screw 14 and allowing appropriate spacing at the distal tip 28 relative to penholder 66 for easy removal of the blank 60 once completed. The bone threads 34 may be created during the 3D printing process or may be machined to specification at a later time. In a third step 86, loose powder is cleared from the part, the part is heat treated to relieve stresses, the part is removed from build plate, and any sacrificial structures are removed (including the penholder 66). In a fourth step 88, any machining and/or surface treatments are performed to complete the final part. For example, the screw head 20 and drive recess 32 may be machined to size, and any threads 34 may be machined, if desired. Other surface treatments such as glass bead blast, k-burr blast, Ti powder blast, anodization, dry and/or wet electropolish may be applied to the implant.

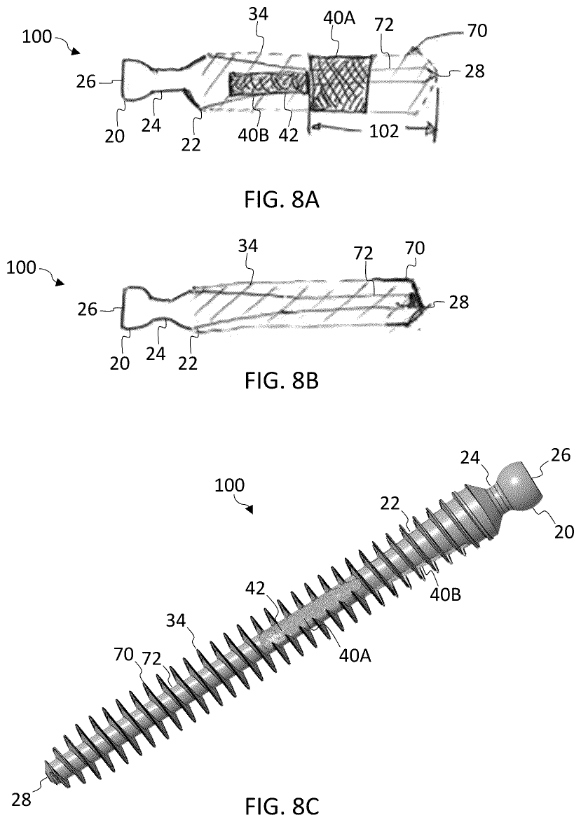

Turning now to FIGS. 8A-8C, a sacroiliac joint implant 100 is shown according to another embodiment. Implant 100 is similar to implant 10 but includes a bone thread 34 with a deep root and an internal taper, and is lacking the outer helical cut 48. No tulip is displayed, but any suitable tulip head assembly may be attached to the screw head 20. In this embodiment, the major outer diameter 70 may be constant while the minor diameter 72 is tapered along its length. The minor diameter 72 may be smallest toward the distal tip 28 and largest toward the proximal end 26. In other words, the SI joint screw 100 may include a tapered shank 22 but with an increasing thread root size resulting in the same screw outer diameter 70 throughout its entire length. Printing a smaller shaft diameter 72 may help in reducing keyhole porosities in the internal geometry by allowing gas to escape to the surface, in turn pores disappearing from the structure. Less keyhole porosities may also help to further increase the structural integrity of the screw 100.

In this embodiment, there may be two windows 40A, 40B that pass through the entire body of the shank 22. Both windows 40A, 40B may be filled with the 3D printed lattice or matrix 42 to promote bone growth. Both windows 40A, 40B may be offset by 90 degrees and set just attached to each other. The intersection of the windows 40A, 40B may be located at a distance 102 from the distal end 28 of the shank 22. For example, the intersection distance 102 may be 50%, 60%, 70% or greater from the distal end 28. As shown in FIG. 8C, the windows 40A, 40B may be shifted proximally along the shank 22 providing for more, deeper threads 34 along the distal portion of the shank 22. The deeper distal threads 34 may increase pull-out strength, thereby anchoring the implant 100 more securely in the bone.

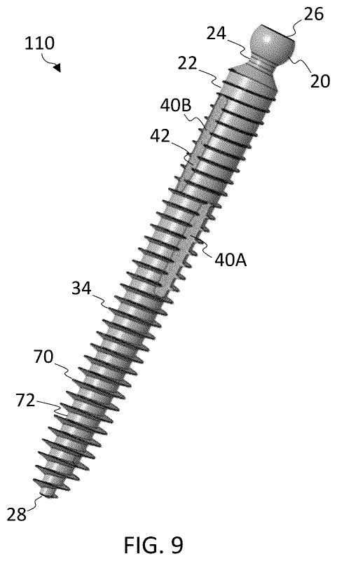

Turning now to FIG. 9, a sacroiliac joint implant 110 is shown according to another embodiment. Implant 110 is similar to implant 100 with a less extreme internal taper resulting in a less extreme thread depth. No tulip is displayed, but any suitable tulip head assembly may be attached to the screw head 20. The major outer diameter 70 may be constant while the minor diameter 72 is tapered along its length. In this embodiment, the degree of taper is less extreme than implant 100, such that the thread depth becomes smaller toward the proximal end 26. The minor diameter 72 may be smallest toward the distal tip 28 and largest toward the proximal end 26 with a gradual taper therebetween. The thread root diameter 72 may be increasing towards the proximal end 28 of the screw 110, which helps the screw bite into the hard bone at the tip 28, and the taper to a shallow rear 26 helps to fixate the screw 110 to prevent windshield-wiping. This geometry may act as a wedge to promote increased initial fixation before fusion can occur.

The locations for the fenestrations 40 may remain the same as implant 100, with the windows 40A, 40B shifted to a more proximal position. Similar to implant 100, the lattice 42 is filled inside internal windows 40A, 40B. The windows 40A, 40B may be set perpendicular to each other and may be joining internally for a larger total combined lattice volume. The matrix 42 may have a similar geometry to cancellous bone, which promotes fusion and bone growth within the screw 110.

In this embodiment, the bone threads 34 may be machined rather than printed to allow for some additional escape for keyhole induced pores. Machined threads 34 may also ensure both more precise manufacturing of the threads 34 and also reduce the porosities that develop along those threads 34 and body by removing an outer layer of material. Outer rough material removal with the addition of an annealing heat treatment and anodization may provide a smooth outer surface that can improve fatigue resistance compared to without these enhancements.

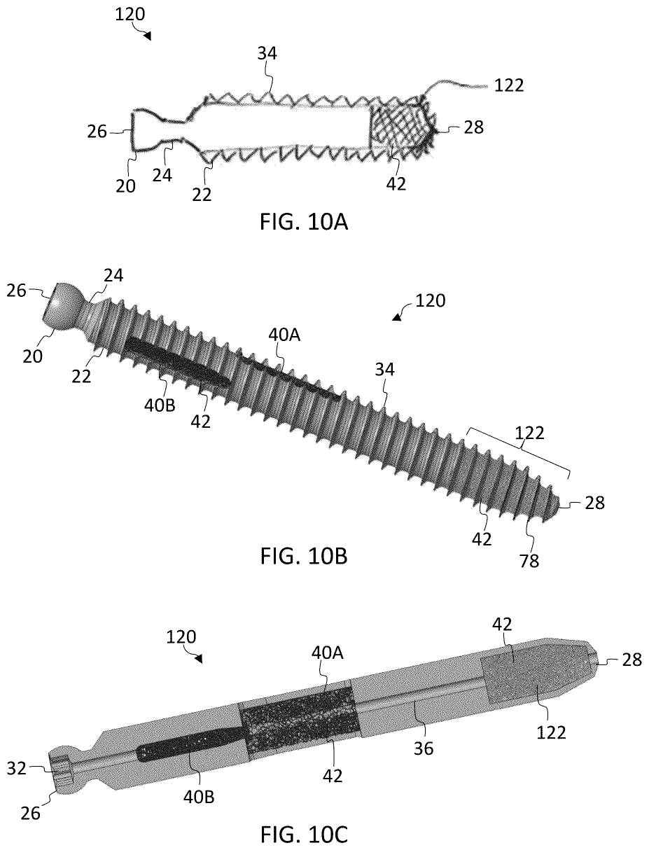

Turning now to FIGS. 10A-10C, a sacroiliac joint implant 120 is shown according to another embodiment. Implant 120 is similar to prior implants with an additional lattice tip 122. No tulip is displayed, but any suitable tulip head assembly may be attached to the screw head 20. In this embodiment, the lattice 42 is located not only in the fenestrated holes/windows 40A, 40B but also towards the distal tip 28 of the screw 120. The additional lattice tip 122 may be located along a distal section of the screw shank 22. For example, the bottom 20 mm of shank 22 (e.g., quarter, fifth, sixth, or less of the shank 22) may include the additional lattice structure 42. The lattice tip 122 may include an internal lattice area 42 provided on the inside and along the shank 22, but not on the exterior threads 34 of the screw 110. The lattice structure 42 along the lattice tip 122 may be the same or different than the lattice structure 42 within the windows 40A, 40B. This configuration provides additional locations for bone in-growth.

In this embodiment, the region of 3D printed porous lattice structure 42 along the lattice tip 122 may be revealed during the thread machining process in post-processing. For example, FIG. 10C shows a blank version of the screw 120, which is 3D printed with additional solid material, before the threads 34 are machined into the shank 22. Once the threads 34 are machined along the shaft 22, the lattice tip 122 is also revealed such that the internal lattice 42 is exposed. For example, the internal lattice 42 may be exposed on the root 76 and/or flanks 74, 80 of the threads 34 while the crests 78 of the threads 34 remain solid. The benefit of this post-machining process is the absence of thread timing that would be needed to match any specifically thread shaped internal pattern or geometry.

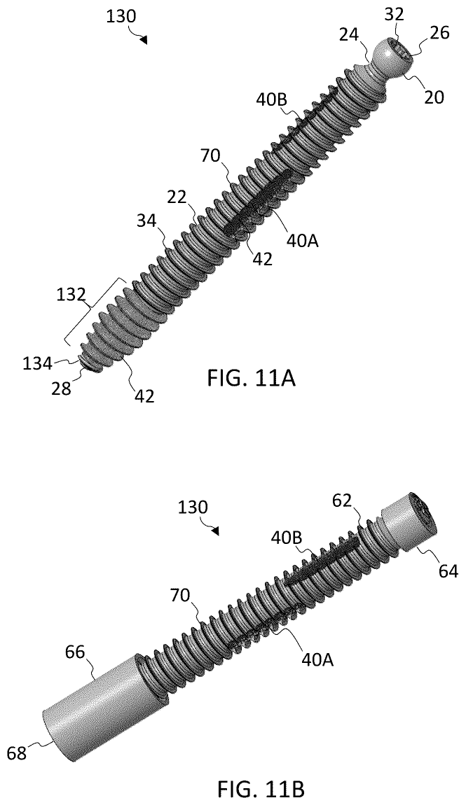

Turning now to FIGS. 11A-11B, a sacroiliac joint implant 130 is shown according to another embodiment. Implant 130 is similar to implant 120 except the lattice structure 42 of the distal lattice tip 132 is fully exposed and a solid front face 134 is added as the distal-most tip. No tulip is displayed, but any suitable tulip head assembly may be attached to the screw head 20. In this embodiment, implant 130 includes exposed distal lattice 132 with lattice structure 42 fully along the threads 34, near the distal tip 28. The exposed lattice structure 42 along the distal lattice tip 132 may encompass up to and including the thread crest 78 as well. The matrix lattice structure 42 provided along the threads 34 of the lattice tip 132 may help to further promote bone in-growth. Printing the lattice 42 on the threads 34 may further help with manufacturing and strength by creating gaps to help with heat distribution and gas escape, avoiding keyhole porosities. In this embodiment, the front portion of the lattice body threads may also be printed with a solid front face 134. In other words, the distal-most front face 134 may be a solid structure instead of the lattice structure 42. The solid front face 134 may help to improve the thread-body strength and shield bone during insertion from the sharp exposed distal tip lattice 132.

As best seen in FIG. 11B, the manufacturing process may include the penholder style of additive manufacturing described previously herein. For example, the penholder 66 may include a small cone of solid material deposited around the implant body to reduce the amount of required support material and to allow for easier removal from the build plate. No mechanical cutting or impaction need be required, which saves time and material usage. Further, the outer diameter 70 of the screw 130 after the additive manufacturing stage remains textured from this printing process. Benefits of the texture may include a slight decrease in insertion torque with comparable pull-out torque for a given size and length. This benefit can decrease surgical fatigue when manually inserting screws while maintaining pull-out resistance. These two approaches may be used for any of the designs described herein.

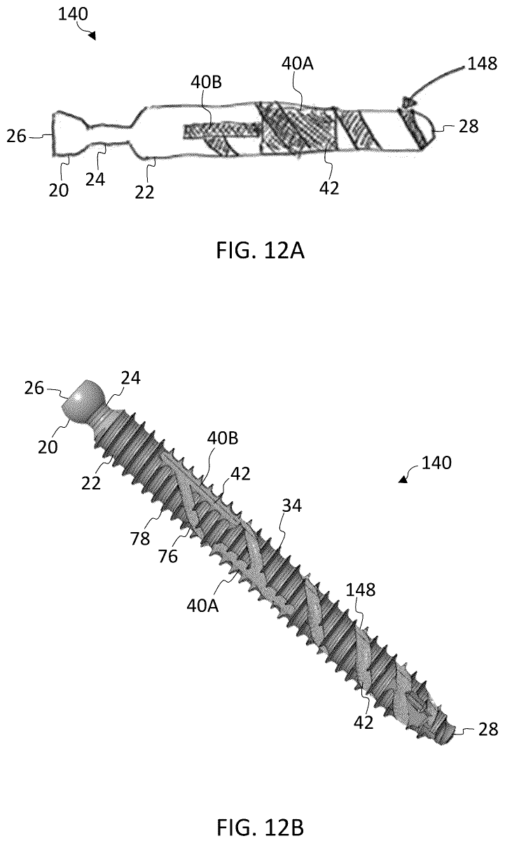

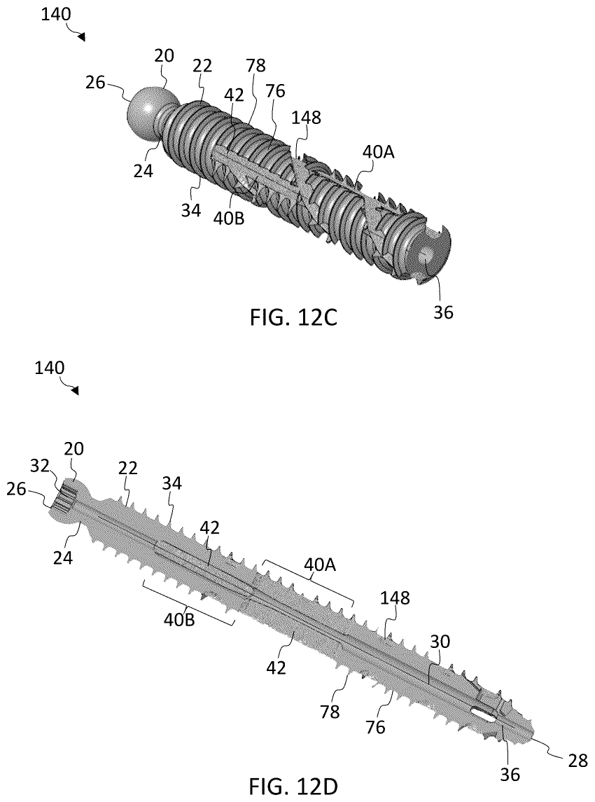

Turning now to FIGS. 12A-12D, a sacroiliac joint implant 140 is shown according to another embodiment. Implant 140 is similar to implant 10 with lattice windows 40A, 40B and a spiral channel 148 filled with internal lattice 42. No tulip is displayed, but any suitable tulip head assembly may be attached to the screw head 20. In this embodiment, the sacroiliac joint fixation implant 140 includes lattice 42 filling the windows 40A, 40B as well as the helical channel 148 to accommodate bone packing and lattice structure. Similar to implant 10, the helical channel 148 may be cut through the screw threads 34. In this embodiment, a continuous spiral groove or channel 148 may be non-coincident with the helical path of the screw threads 34, thereby interrupting the screw threads 34. The continuous helical cut 148 may extend from the proximal window 40B to the distal end 28 of the implant 140. The pitch of the helical cut 148 may be greater or larger than the pitch of the screw threads 34 such that it overlaps the path, for example, five or more regular bone threads 34, or six or more regular bone threads 34 on the screw's body. In this embodiment, the depth of the helical cut 148 may be equal to the root 76 of the screw threads 34 or deeper than the root 76 such that the screw threads 34 are completely eliminated or erased along the helical cut 148. By having the lattice matrix 42 throughout the majority of the screw's length, bone growth may be fostered along the entire screw rather than only in certain sections. In one embodiment, for the top half of the screw 140, the shank 22 may be constructed with the internal lattice structure 42, while the bottom half only has the lattice 42 on the surface of the shank 22, for example, along the helical cut 148. Front cutting flute regions and channels may also be provided to allow for internal bone packing when a k-wire is not used. More cuts into the surface of the screw 140 allow for gaseous pores to escape to the top of the material, removing pores and stress concentrations in the material.

With further emphasis on FIGS. 12C and 12D, an axial cross section (FIG. 12C) and longitudinal cross section (FIG. 12D) of the implant 140 are shown after additive manufacturing and post processing. As previously described for FIGS. 5A-5B, a penholder style of additive manufacturing may be utilized where a small cone of solid material 66 is deposited around the implant body to reduce the amount of required support material and to allow for easier removal from the build plate. The penholder 66 may remove the need for mechanical cutting and impaction, thereby providing for savings of time and material usage. Further, the outer diameter of the screw 140 after the additive manufacturing stage may remain textured from the 3D printing process. Benefits of the texture may include a slight decrease in insertion torque with comparable pull-out torque for a given size and length. This benefit can decrease surgical fatigue when manually inserting screws while maintaining pull-out resistance. These approaches can be used for any of the designs described herein.

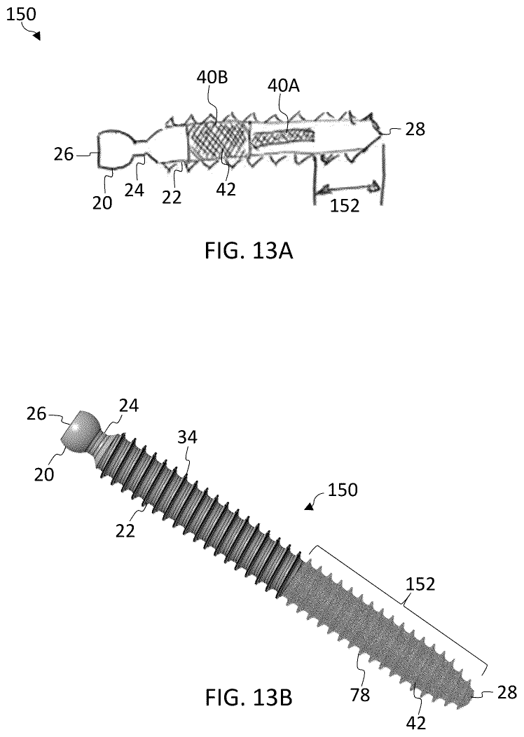

Turning now to FIGS. 13A-13B, a sacroiliac joint implant 150 is shown according to another embodiment. Implant 150 is similar to implant 120 and includes the lattice structure 42 throughout the entire distal tip 152 including the threads 34 and includes a larger volume of the screw body. No tulip is displayed, but any suitable tulip head assembly may be attached to the screw head 20. The additional lattice tip 152 may be located along a distal portion of the screw shank 22. For example, the bottom 43 mm of shank 22 (e.g., half or less of the shank 22) may include the lattice structure 42. The lattice tip 152 may include a lattice area 42 extending fully through the tip of the implant 150, which includes the bone threads 34 of the screw 150. The lattice structure 42 along the lattice tip 152 may be the same or different than the lattice structure 42 within the windows 40A, 40B, if present. FIG. 13A shows one embodiment with the presence of windows 40A, 40B with integral lattice structure 42, and FIG. 13B shows another embodiment with the windows 40A, 40B omitted. These configurations may provide for additional locations for bone in-growth, which may be desirable for the intended use and placement of the device.

In the embodiment shown in FIG. 13B, the half closer to the driver end 26 may be provided without any lattice structure 42 to provide increased strength to fixate in the bone and to provide a longer fatigue life than a fully-lattice-printed screw. The lattice tip 152 may be made completely of lattice matrix 42 to provide more fusion in the cancellous region of the bone. In one embodiment, the implant 150 may be formed of a multi-part construction. For example, the two halves of the screw 150 may be welded together, with the distal lattice tip 152 being formed via 3D printing and the proximal end being created by traditional machining. By only 3D printing a smaller portion of the screw (e.g., only the lattice tip 152), the hybrid manufacturing process may help to minimize the chance for pores to develop in the critical areas for stress concentrations.

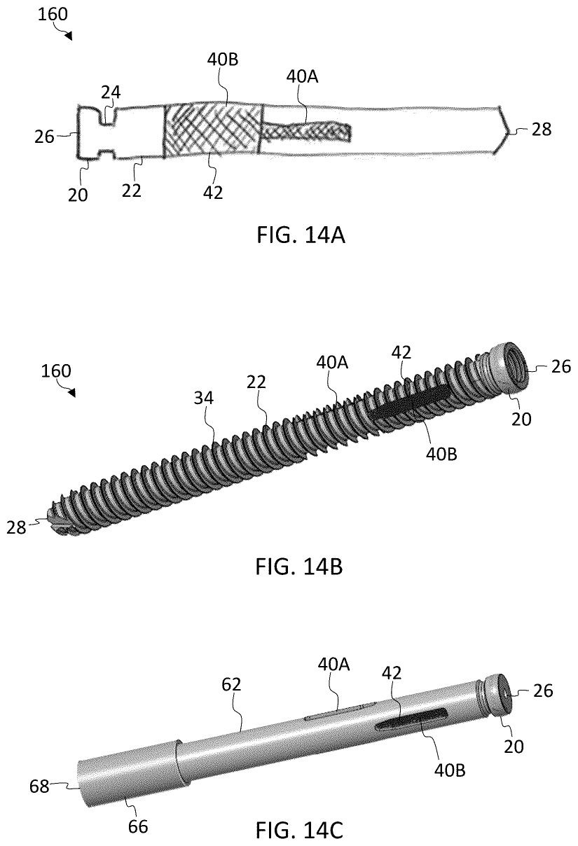

Turning now to FIGS. 14A-14C, a sacroiliac joint implant 160 is shown according to another embodiment. Implant 160 is similar to prior implants with lattice filled windows 40A, 40B, but with an otherwise solid shank construction. Following 3D printing, the outer threads 34 may be fully machined for porosity minimization and precision benefits. This version of the implant 160 intentionally does not receive a tulip and is intended to sit below a rod construct mating style of implant. This allows the surgical technique to achieve a close trajectory to the S2AI style screw, but to sit lower than a tulip and reduce interference at the proximal end 26. Additional points of fixation promote increased construct rigidity and additional lattice regions 40A, 40B across the SI joint promote a larger fusion mass. The cannulation 36 may be maintained throughout the entire length of the screw 160.

As best seen in FIG. 14C, the penholder style of additive manufacturing may be used during construction of implant 160. The penholder 66 may include a small cone of solid material deposited around the distal tip 28 of the implant 160 to reduce the amount of required support material and to allow for easier removal from the build plate. The penholder 66 may eliminate the need for mechanical cutting or impaction, thereby reducing both time and material consumption. The bone threads 34 may be machined following the additive manufacturing process for precision threads. Further, the outer diameter of the screw 160, after the additive manufacturing stage, may remain textured from the 3D printing process. The texture may provide for a slight decrease in the torque required for insertion, which can decrease surgical fatigue when manually inserting screws while maintaining pull-out resistance.

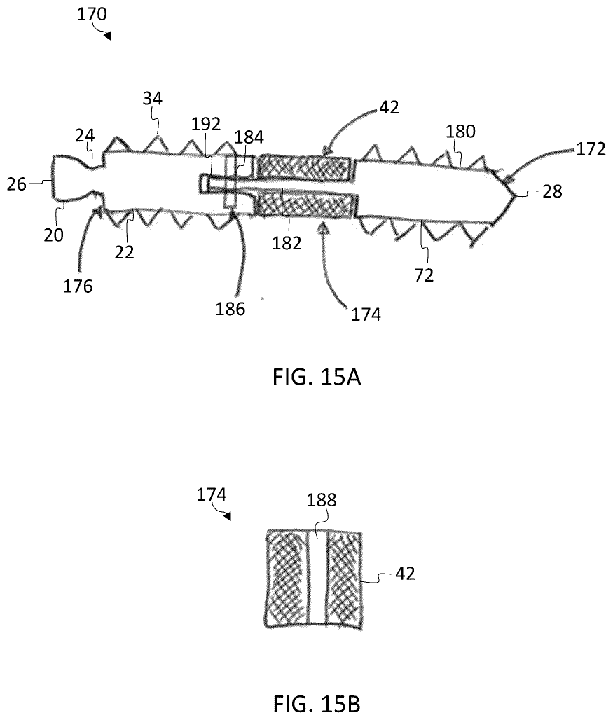

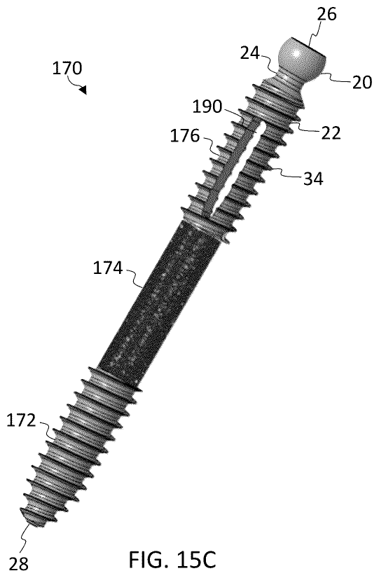

Turning now to FIGS. 15A-15C, a sacroiliac joint implant 170 is shown according to another embodiment. In this embodiment, the implant 170 may be assembled from three distinct parts: a solid tip 172, a lattice core 174, and a solid base 176. The lattice core 174 is mounted to the middle section of a traditionally manufactured screw shank, for example, made using extruded stock. This approach combines the mechanical benefits of extruded stock with the bony in-growth benefits of an additively manufactured structure. No tulip is displayed, but any suitable tulip head assembly may be attached to the screw head 20.

The solid tip 172 includes a distal tip portion 180 with bone threads 34. The distal tip portion 180 terminates as the distal-most tip 28 to engage bone. The proximal end of the distal tip portion 180 includes an extension 182 that extends toward the proximal end 26 of the implant 170. The extension 182 may include a cylindrical body extending along the central longitudinal screw axis 30. The extension 182 may define a proximal transverse opening 184 for receiving an assembly pin 186. The solid tip 172 may be created by traditional manufacturing, for example, using extruded stock.

The lattice core 174 may include a ring of lattice matrix 42 with a central through opening 188 extending therethrough. The central opening 188 may be sized and dimensioned to receive the extension 182 therethrough. The outer diameter of the lattice core 174 may be the same or similar to the outer dimensions of the solid tip 172 and/or the solid base 176. For example, the outer diameter of the core 174 may match the minor diameter 72 of the shaft 22. The lattice core 174 may be 3D printed, for example, using the additive laser powder bed fusion process to provide the lattice structure 42 through the body of the lattice core 174.

The solid base 176 may include a portion of the shaft 22 with bone threads 34. The solid base 176 includes the screw head 20 configured for receiving a tulip assembly. As shown in FIG. 15C, the shaft portion 22 may include a fenestration or longitudinal window 190, for example, similar to the shape of window 40. In this embodiment, however, the window 190 may be completely open without any lattice structure. A distal portion of the solid base 176 defines a recess or channel 192 sized and dimensioned to receive the proximal-most end of the extension 182. The assembly pin 186 may be configured to secure the extension 182 within the solid base 176, thereby securing the lattice core 174 between the solid tip 172 and solid base 176. It will be appreciated that the extension/pin assembly may be reversed or reconfigured between the components or an alternative method of attachment may be used. The solid base 172 may be formed by traditional manufacturing, for example, using extruded stock. It will be further appreciated that the lattice and solid components may be reordered or modified as desired. For example, the tip 172 and/or base 176 may be constructed from 3D manufacturing while the core 174 is constructed via traditional manufacturing processes or in any other suitable combination. Furthermore, additional sections or components may be added to the shaft 22, for example, creating further lattice areas or in-growth opportunities.

In one embodiment, the inner core 174 may be welded around the screw shank 22 to form a fully cylindrical lattice structure. Other attachment methods may also be used, such as a snap finger type feature or thread. The implant 170 may be assembled in three parts with a weld. The region to be welded may have an internal solid metal border to promote weld pool consistency. Dividing the part into three sections 172, 174, 176 may help with greatly reducing the chances of pores developing in the printed material as the only printed portion 174 exhibits many gaps allowing for gas to escape. The machined parts 172, 176 reduce the chance of stress concentrations forming, giving the printed material 174 more structural integrity.

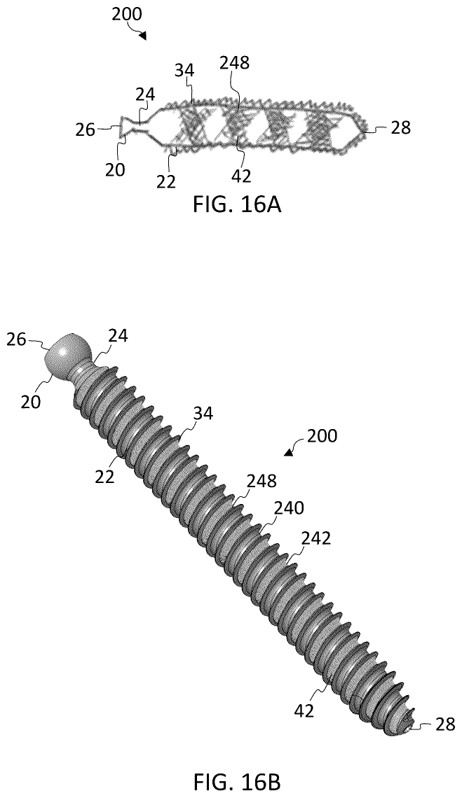

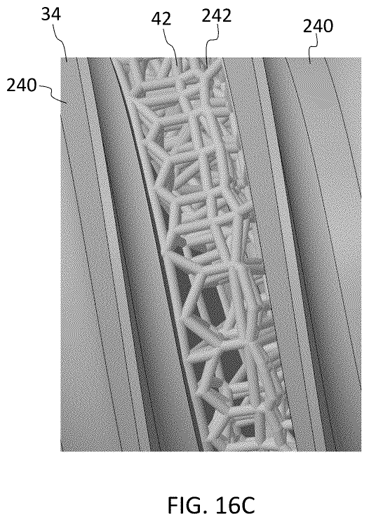

Turning now to FIGS. 16A-16C, a sacroiliac joint implant 200 is shown according to another embodiment. In this embodiment, the implant 170 includes a helical path 248 filled with lattice 42, similar to helical cut 48. In this embodiment, however, the helical path 248 follows the same path as the bone threads 34. In other words, the helical path 248 may be coincident with the threads 34 and does not disrupt the thread pathway. The bone thread 34 may include a double start thread with a first lead 240 and a second lead 242. The helical path 248 filled with lattice 24 may follow one lead 242 while the alternate lead 240 remains unaltered and solid. In other words, the lattice 42 may run down all throughout the length of the screw shank 22 along one of the thread grooves while the other thread remains intact. FIG. 16C shows a close-up side view of the lattice threaded section 242 between solid threads 240. The double start thread pattern may offer a higher load capacity as the two threads support each other.

In one embodiment, the lattice 42 may only be provided at the outer surface and may not extend throughout the entire diameter of the shank 22 to maintain core structural integrity. The even distribution of lattice 42 throughout the screw 200 may help to create a uniform distribution of surfaces for small keyhole porosities to escape, preventing the development of stress concentrations from said keyhole porosities. The lattice 42 may extend throughout the length of the screw 200 such that there are no graft windows to avoid further reducing structural integrity of the design. The lattice 42 permeating the length of the screw 22 allows for sufficient bone in-growth opportunities and fusion.

The entire screw 200 may be created using 3D additive manufacturing. The outer diameter of the screw 200, after the additive manufacturing stage, may remain textured from this printing process. This, along with the lattice matrix surfacing along the length of the shank 22 may help to decrease insertion torque making for an easier procedure for the operator than a double start thread would normally entail. The screw head 20, drive feature 32, and/or neck 24 may be machined onto the implant 200 in post-processing. Although no tulip head assembly is shown, any appropriate tulip head can be affixed to the screw head 20 during use.

The implants described herein allow for fixation and fusion of the SI joint using threaded implants that incorporate additively manufactured geometry for improved fusion properties. The features may help to promote boney in-growth, improve anti-haloing, encourage initial screw purchase, and provide better fusion properties compared to current lumbosacral polyaxial screw technology that can be used in a SAI style technique. Other advantages of additively manufactured screws include the ability to obtain fixation and fusion properties from the same implant, while allowing attachment to a rod and screw construct. Additively manufactured implants also allow for inclusion of biologically relevant lattice structure that promotes fusion, which cannot be made using traditional manufacturing approaches.

In some embodiments, interchangeable components and/or instrumentation may be provided. This may help to reduce the number of sets required in the operating room and to streamline the technique. Using instrumentation across platforms further reduces the manufacturing burden by reducing the number of new instruments required.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. An implant comprising:
a tulip head having two arms defining a rod slot therebetween; and
a bone fastener extending along a central longitudinal axis including a screw shank with bone threads following a helical path, a distal tip configured to facilitate insertion into bone, and a proximal end having a screw head receivable in the tulip head,
wherein the screw shank defines first and second longitudinal windows extending therethrough, the first and second longitudinal windows are staggered and shifted longitudinally and rotationally relative to one another, the screw shank further defines a helical cut that is non-coincident with the helical path of the bone threads such that the helical cut interrupts at least some of the bone threads, wherein the first and second longitudinal windows and the helical cut are filled with a lattice structure configured for promoting bone ingrowth.

2. The implant of claim 1, wherein the first and second longitudinal windows are rotated 90 degrees around the central longitudinal axis of the screw shank relative to one another.

3. The implant of claim 1, wherein the first and second longitudinal windows are obround slots.

4. The implant of claim 1, wherein the first and second longitudinal windows intersect such that peripheries of the windows overlap with one another.

5. The implant of claim 1, wherein the helical cut includes a first distal helical segment that overlaps a portion of the first longitudinal window and a second proximal helical segment that overlaps a portion of the second longitudinal window.

6. The implant of claim 1, wherein a pitch of the helical cut is greater than the pitch of the bone threads.

7. The implant of claim 1, wherein a depth of the helical cut is shallower than a root of the screw threads.

8. The implant of claim 1, wherein the screw threads include an asymmetrical profile with a sloped leading edge and a flat trailing edge.

9. The implant of claim 1, wherein the lattice structure includes interconnected struts defining open pores of different sizes.

10. A manufacturing process comprising:
applying a layer of fine metal powder to a build plate of a three-dimensional printing machine and selectively sintering metal in prescribed locations to create a layer of a screw part blank; and
consecutively adding and sintering layers of fine metal power to prior layers to build up the screw part blank, wherein the screw part blank includes a screw shank with solid and lattice portions, the solid portion includes the bone threads, and the lattice portions fill first and second longitudinal transverse windows and a helical cut about a periphery of the screw shank, wherein the first and second longitudinal transverse windows are staggered and shifted longitudinally and rotationally relative to one another, and the helical cut does not align with a helical path of the bone threads, thereby interrupting some of the bone threads where the paths intersect.

11. The process of claim 10 further comprising creating a penholder at a distal tip of the screw part blank when adding and sintering the layers of metal powder.

12. The process of claim 11, wherein the penholder includes a cone of solid material deposited around the distal tip of the screw part blank.

13. The process of claim 11 further comprising creating space at the distal tip relative to the penholder for easy removal of the screw part blank from the penholder.

14. The process of claim 10, wherein the bone threads include a tapered buttress style geometry where inter-thread support material is not needed and the threads are able to self-support during the entire manufacturing process.

15. The process of claim 10, wherein the three-dimensional printing machine includes a laser powder bed fusion machine.

16. The process of claim 10 further comprising machining the screw part blank to form the screw head and drive recess, thereby creating the final bone screw.

17. A method for stabilizing a sacroiliac joint, the method comprising:
providing an implant having a screw shank with bone threads, the screw shank defining first and second longitudinal windows extending therethrough that are staggered and shifted longitudinally and rotationally relative to one another, the screw shank further defining a helical cut that has a pitch greater than a pitch of the bone threads such that the helical cut interrupts at least some of the bone threads, wherein the first and second longitudinal windows and the helical cut are filled with a lattice structure that acts as a scaffold for bone healing and bone interdigitation;
accessing a sacrum and/or ilium of a patient; and
inserting the implant across the sacroiliac joint such that once the implant is fully seated, the first and second longitudinal windows engage with the sacrum and ilium, respectively, thereby traversing the sacroiliac joint at final placement to increase the likelihood of fusion.

18. The method of claim 17, wherein the first longitudinal window is a distal window configured to be positioned in the ilium, and the second longitudinal window is a proximal window configured to be positioned in the sacrum, and an intersection of the windows provides for in-growth capabilities through the lattice structure, further promoting fusion.

19. The method of claim 17, wherein a pair of implants are used as bilateral S2-alar-iliac screws to fix the sacrum to the ilium in a lumbosacral fixation.

20. The method of claim 17 further comprising accessing the sacroiliac joint with a robotic and navigational system.
